(19) United States
(12) Patent Application Publication
(10) Pub. No.: US 2018/0220051 A1
(43) Pub. Date: Aug. 2, 2018

(54) ENDOSCOPE, IMAGE PICKUP MODULE, AND MANUFACTURING METHOD OF IMAGE PICKUP MODULE

(71) Applicant: OLYMPUS CORPORATION, Tokyo (JP)

(72) Inventor: Jumpei YONEYAMA, Kamiina-gun (JP)

(73) Assignee: OLYMPUS CORPORATION, Tokyo (JP)

(21) Appl. No.: 15/935,210

(22) Filed: Mar. 26, 2018

Related U.S. Application Data
(63) Continuation of application No. PCT/JP2015/077717, filed on Sep. 30, 2015.

(51) Int. Cl. H04N 5/225; A61B 1/00; A61B 1/018; A61B 1/04

(52) U.S. Cl. CPC H04N 5/2253; A61B 1/0011; H04N 2005/2255; A61B 1/04; A61B 1/00124; A61B 1/018

(57) ABSTRACT

An endoscope includes an image pickup module placed in a distal end portion, the image pickup module including an image pickup device to which a cover glass is bonded, a positioning bump placed on the image pickup device, and a recessed portion formed in the cover glass, an upper portion of the positioning bump being inserted into the recessed portion, and a relative position in three axial directions of the image pickup device and the cover glass being defined by an abutment position of the positioning bump and the recessed portion.

### CROSS REFERENCE TO RELATED APPLICATION

This application is a continuation application of PCT/JP2015/077717 filed on Sep. 30, 2015, the entire contents of which are incorporated herein by this reference.

### BACKGROUND OF INVENTION

### 1. Field of the Invention

The present invention relates to an endoscope having an image pickup module in which an optical member is bonded to a light receiving surface of an image pickup device, the image pickup module in which the optical member is bonded to the light receiving surface of the image pickup device, and a manufacturing method of the image pickup module in which the optical member is bonded on the light receiving surface of the image pickup device.

### 2. Description of the Related Art

A large number of image pickup devices each having a small size in plan view can be collectively produced by forming a number of light receiving portions and the like on a semiconductor wafer, and cutting and separating the light receiving portions into individual pieces. An optical member such as a cover glass is bonded to a light receiving surface of the image pickup device which is separated into an individual piece.

Japanese Patent Application Laid-Open Publication No. 2002-343949 discloses using a dummy bump exclusively for positioning as height adjustment means in order to ensure accuracy in positioning in the vertical direction.

### SUMMARY OF THE INVENTION

An endoscope of an embodiment of the present invention is an endoscope including an image pickup module in a distal end portion of an insertion portion, wherein the image pickup module includes an optical member, and an image pickup device to which the optical member is bonded via a bonding layer to cover a light receiving portion so that light is incident on the light receiving portion via the optical member, a positioning bump is placed in a peripheral portion of the light receiving portion of the image pickup device, a recessed portion is formed in the optical member, and an upper portion of the positioning bump of the image pickup device is inserted into the recessed portion of the optical member, and a relative position in three axial directions of the image pickup device and the optical member is defined by an abutment position of the positioning bump and the recessed portion.

Further, an image pickup module of an embodiment is an image pickup module including an optical member, and an image pickup device to which the optical member is bonded via a bonding layer to cover a light receiving portion so that light is incident on the light receiving portion via the optical member, wherein a positioning bump is placed in a peripheral portion of the light receiving portion of the image pickup device, a recessed portion is formed in the optical member, and an upper portion of the positioning bump of the image pickup device is inserted into the recessed portion of the optical member, and a relative position in three axial directions of the image pickup device and the optical member is defined by an abutment position of the positioning bump and the recessed portion.

Further, a manufacturing method of an image pickup module of an embodiment includes a step of producing an optical member in which recessed portions are formed, and an image pickup device in which a plurality of positioning bumps are placed on a peripheral portion of a light receiving portion, a step of a relative position in three axial directions of the image pickup device and the optical member being positioned by inserting upper portions of the positioning bumps of the image pickup device into the recessed portions of the optical member, and a step of curing an adhesive agent interposed between the optical member and the image pickup device, in a positioned state.

### BRIEF DESCRIPTION OF THE DRAWINGS

FIG. 1 is an exploded view of an image pickup module of a first embodiment;

FIG. 2 is a perspective view of a positioning bump of the image pickup module of the first embodiment;

FIG. 3 is a sectional view showing a relationship between the positioning bump and a recessed portion of the image pickup module of the first embodiment;

FIG. 4A is a perspective view of a positioning bump of an image pickup module of modification 1 of the first embodiment;

FIG. 4B is a sectional view showing a relationship between a positioning bump and a recessed portion of an image pickup module of modification 2 of the first embodiment;

FIG. 5A is a sectional view showing a relationship between a positioning bump and a recessed portion of an image pickup module of modification 3 of the first embodiment;

FIG. 5B is a sectional view showing a relationship between a positioning bump and a recessed portion of an image pickup module of modification 4 of the first embodiment;

FIG. 5C is a sectional view showing a relationship between a positioning bump and a recessed portion of an image pickup module of modification 5 of the first embodiment;

FIG. 5D is a sectional view showing a relationship between a positioning bump and a recessed portion of an image pickup module of modification 6 of the first embodiment;

FIG. 5E is a sectional view showing a relationship between a positioning bump and a recessed portion of an image pickup module of modification 7 of the first embodiment;

FIG. 6A is a perspective view of a positioning bump of an image pickup module of modification 8 of the first embodiment;

FIG. 6B is a top view showing a relationship between the positioning bump and a recessed portion of the image pickup module of modification 8 of the first embodiment;

FIG. 7 is a perspective view of a cover glass of an image pickup module of modification 9 of the first embodiment;

FIG. 8A is a sectional view of an image pickup module of modification 10 of the first embodiment;

FIG. 8B is a sectional view of an image pickup module of modification 11 of the first embodiment;

FIG. 8C is a sectional view of an image pickup module of modification 12 of the first embodiment;

FIG. 9 is a sectional view of an image pickup module of a second embodiment;

FIG. 10 is a perspective view of an image pickup device of an image pickup module of modification 1 of the second embodiment; and

FIG. 11 is a perspective view of an endoscope system including an endoscope of the embodiments.

### DETAILED DESCRIPTION OF THE PREFERRED EMBODIMENTS

### First Embodiment

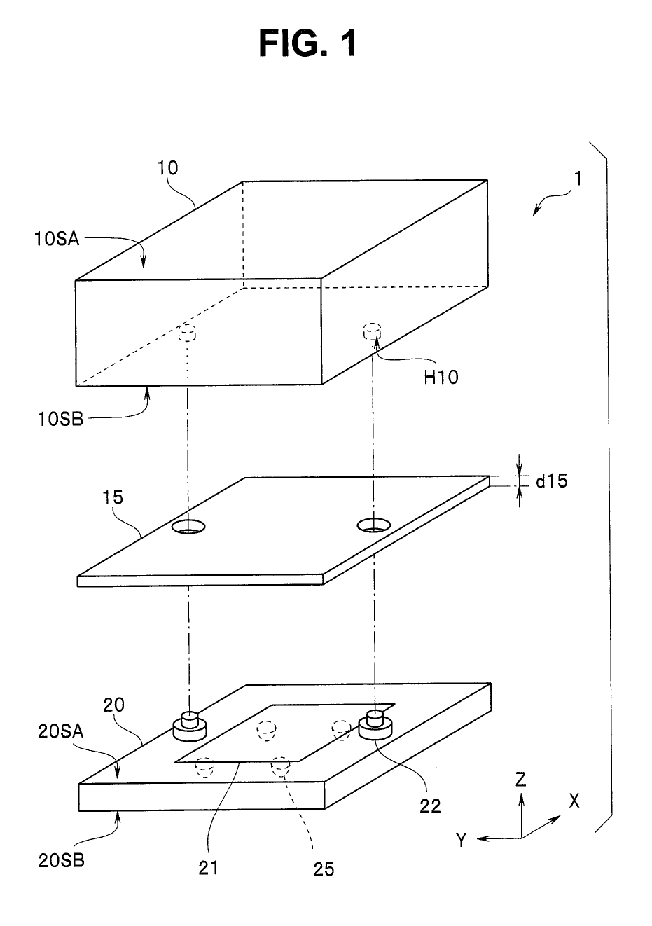

As shown in FIG. 1, an image pickup module 1 of the present embodiment includes a cover glass 10 that is an optical member, an image pickup device 20, and a bonding layer 15.

Note that in the following explanation, drawings based on respective embodiments are schematic, and attention should be paid to the fact that relationships between thicknesses and widths of respective portions, ratios of thicknesses of the respective portions, relative angles and the like differ from actual relationships, ratios, relative angles and the like, and parts where mutual size relationships and ratios differ may be included among the drawings. Further, in the orthogonal coordinate system including an X-axis, a Y-axis and a Z-axis, a Z-axis direction is set as a vertical direction, and an X-axis direction is referred to as an X-direction, for example.

The cover glass 10 is a rectangular parallelepiped transparent member having a dimension in plan view (an XY in-plane dimension) of 2 mm square and a thickness (a Z-direction dimension) of 400 μm. The transparent member may be a rectangular parallelepiped resin member.

The image pickup device 20 has, for example, a thickness of 300 μm, and a dimension in plan view of 2.5 mm by 2.5 mm. On a light receiving surface 20SA of the image pickup device 20, a rectangular light receiving portion 21 of 1.8 mm square is formed. On a back surface 20SB facing the light receiving surface 20SA, an external electrode terminal 25 electrically connected to the light receiving portion 21 via through wiring (not illustrated) or the like is placed.

The image pickup device 20 is produced by cutting a semiconductor wafer on which a plurality of image pickup devices (CCD or CMOS devices) are produced.

Note that in the image pickup module 1, the dimension in plan view of the cover glass 10 and a dimension in plan view of the image pickup device 20 are substantially the same, but the cover glass 10 may be larger than the image pickup device 20, or the image pickup device 20 may be larger than the cover glass 10.

The cover glass 10 and the image pickup device 20 are bonded via the transparent bonding layer 15 of a thickness of d15 (μm). That is, the cover glass 10 is bonded via the bonding layer 15 so as to cover the light receiving portion 21 so that light is incident on the light receiving portion 21 through the cover glass 10. The bonding layer 15 is, for example, an ultraviolet curable resin or a thermosetting resin, and is liquid before cured. Note that, in a case of an image pickup device in which a plurality of micro-lenses are placed on the light receiving portion 21, the cover glass 10 may be bonded via a frame-shaped light shielding bonding layer placed in a periphery of the light receiving portion 21.

Two positioning bumps 22 are placed in peripheral portions of the light receiving portion 21 of the image pickup device 20. Hereinafter, “the positioning bump” will be simply called “the bump”. Note that the bump 22 is placed on a metal film pad (not illustrated).

Two recessed portions H10 are formed on a back surface 10SB facing a top surface (light incident surface) 10SA of the cover glass 10. An upper portion of the bump 22 is inserted into the recessed portion H10 of the cover glass 10.

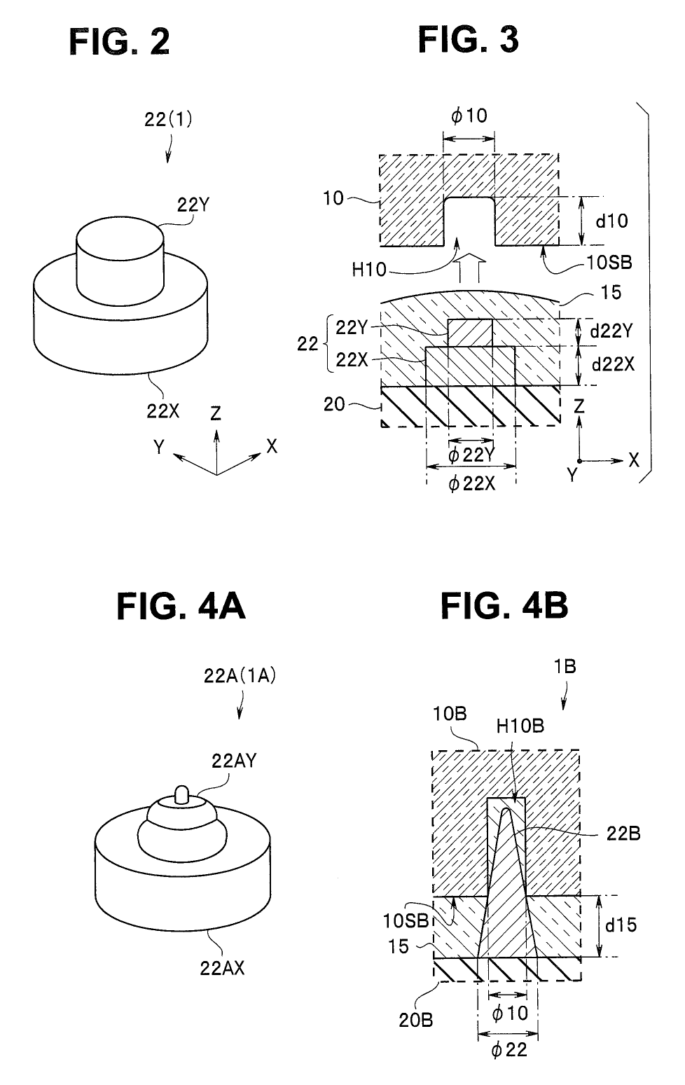

As shown in FIG. 2 and FIG. 3, the bump 22 of the image pickup module 1 is a two-stage bump including a lower bump 22X and an upper bump 22Y. An external dimension (an outside diameter) φ22Y of the upper bump 22Y is smaller than an external dimension (an outside diameter) φ22X of the lower bump 22X.

The bump 22 is a two-stage plated bump in a circular-column shape with a height of one stage of 20 μm to 100 μm, for example. The upper bump 22Y may be in a conical shape if only a section is circular. Further, the recessed portion H10 is circular in section, for example, and has a depth d10 of 20 μm to 200 μm.

The bump 22 may be a multistage bump with three stages or more, may be a stud bump, a ball bump or the like, or may be a combination of a plated bump, a stud bump, a ball bump and the like as described later. Further, the bump 22 may be placed by using a 3D printer.

The recessed portion H10 is formed by wet etching or dry etching by using an etching mask by photolithography. After a plurality of recessed portions H10 are formed on a glass wafer including a plurality of cover glasses 10, the glass wafer is cut and separated into pieces, and thereby a large number of cover glasses 10 in which the recessed portions are formed can be produced. Further, the recessed portion H10 may be formed by mechanical processing, for example, drilling.

An internal dimension (an inside diameter) φ10 of the recessed portion H10 is larger than the external dimension φ22Y of the upper bump 22Y, and is smaller than the external dimension φ22X of the lower bump 22X. Further, a height d22Y of the upper bump 22Y is shorter than the depth d10 of the recessed portion H10.

Consequently, the bump 22 has only the upper bump 22Y inserted into the recessed portion H10, and the back surface 10SB of the cover glass 10 abuts on a top surface of the lower bump 22X.

The two bumps 22 are placed on the image pickup device 20, and when the respective bumps 22 are inserted into the recessed portion H10 of the cover glass 10, a relative position of the cover glass 10 and the image pickup device 20 is positioned in three axial directions of an X axis, a Y axis and a Z axis, that is, not only in the in-plane direction (the XY-direction) parallel with the light receiving surface 20SA, but also in the vertical direction (the Z-direction).

A relative position in the in-plane direction of the cover glass 10 and the image pickup device 20 is defined by abutment positions of the respective upper bumps 22Y that are inserted into the two recessed portions H10. Note that the internal dimension (the inside diameter) φ10 of the recessed portion H10 and the external dimension φ22Y of the upper bump 22Y may be the same, that is, the recessed portion H10 and the upper bump 22Y may be tightly fitted, but insertion becomes difficult. Consequently, in order to fit the recessed portion H10 and the upper bump 22Y loosely, it is preferable to make setting so that a gap of approximately 5 μm to 50 μm, that is, so-called “play (backlash)” occurs between both of the recessed portion H10 and the upper bump 22Y in the in-plane direction. The backlash is an error in positioning in the in-plane direction, but the aforementioned range is an allowable range corresponding to specifications of the image pickup module 1.

Note that in a case of the upper bump 22Y formed from a soft metal such as gold, the upper bump 22Y is pressed and inserted into the recessed portion H10, and thereby is plastically deformed to be tightly fitted to the recessed portion H10, even when the external dimension φ22Y is larger than the internal dimension φ10 of the recessed portion H10.

A distance between the back surface 10SB and the light receiving surface 20SA that are in relative positions in the vertical direction, that is, a thickness d15 of the bonding layer 15 is accurately defined by a height d22X of the lower bump 22X. In other words, the thickness d15 of the bonding layer 15 is defined by a top surface position of the lower bump 22X which is an abutment position of the bump 22 and the recessed portion H10.

The upper bump 22Y is inserted into the recessed portion H10 of the cover glass 10, and in a state where the back surface 10SB of the cover glass 10 and the top surface of the lower bump 22X are pressed to abut on each other, curing treatment of the bonding layer 15, for example, ultraviolet irradiation is performed, whereby no fear of the position of the cover glass 10 moving by the curing treatment exists.

That is, a manufacturing method of the image pickup module of the embodiment includes a step of producing an optical member, and an image pickup device in which a plurality of positioning bumps are placed in a peripheral portion of a light receiving portion, a step of the optical member covering the light receiving portion of the image pickup device, and being positioned by inserting upper portions of the positioning bumps into the recessed portions of the optical member, and a step of curing an adhesive agent interposed between the optical member and the image pickup device in a positioned state.

In a case of an ultra-small image pickup device, it is not easy to position an optical member accurately and bond the optical member. For example, when a cover glass of 1.8 mm square is bonded to an image pickup device with a light receiving surface of 2 mm by 3 mm, and a light receiving portion of 1.5 mm square, positioning precision in the in-plane direction of 0.05 mm or less is required. Further, the position may be deviated by shrinkage of the adhesive agent at the time of performing curing treatment of the adhesive agent.

Further, positioning in not only the in-plane direction but also in the vertical direction, that is, accuracy in a distance between the back surface of the cover glass and the light receiving surface of the image pickup device and parallelism of both the back surface of the cover glass and the light receiving surface of the image pickup device is sometimes required.

However, when at least two sets of positioning portions including the bumps 22 and the recessed portions H10 are included, the cover glass 10 can be positioned to the image pickup device 20, in three axial directions of X, Y and Z easily and accurately. That is, in the image pickup module 1, the cover glass 10 is accurately positioned and bonded to the light receiving surface 20SA of the image pickup device 20.

Note that a semiconductor wafer on which a plurality of image pickup devices 20 are formed is cut into a device group in which the plurality of image pickup devices 20 are connected in a lateral direction (a Y direction), positioning/bonding of an elongated cover glass is performed by using positioning bumps of any one image pickup device or dummy image pickup device included in the element group, and thereafter, the device group may be separated into the individual image pickup devices 20. In the manufacturing method, the positioning bumps do not have to be placed on all of the image pickup devices.

### <Modifications of First Embodiment>

Next, image pickup modules of modifications of the first embodiment will be described. The image pickup modules of the modifications are analogous to the image pickup module 1, and have same effects as the effect of the image pickup module 1, so that the same components will be assigned with the same reference signs, and explanation will be omitted.

### <Modification 1>

A bump 22A of an image pickup module 1A of modification 1 shown in FIG. 4A is a multistage bump in which a gold stud bump is placed as an upper bump 22AY on a top surface of a lower bump 22AX which is a plated bump. An external dimension of the upper bump 22AY is smaller than an external dimension of the lower bump 22AX. Note that the positioning bump may be a multistage bump with three stages or more, both of an upper portion and a lower portion may be stud bumps, or the upper portion may be a ball bump.

The bump 22A is easier to produce than the bump 22.

### <Modification 2>

A bump 22B of an image pickup device 20B of an image pickup module 1B of modification 2 shown in FIG. 4B is substantially conical. That is, the bump 22B is an inclined bump in which an external dimension of an upper portion continuously becomes smaller than an external dimension of a lower portion. An external dimension φ22 of the lower portion of the bump 22B is larger than an opening internal dimension φ10 of a recessed portion H10B in which the bump 22B is inserted.

Consequently, when the bump 22B is inserted into the recessed portion H10B, the bump 22B abuts on the recessed portion H10B at a position where an outside diameter of the bump 22B becomes same as an opening diameter of the recessed portion H10B, and is tightly fixed. That is, a thickness d15 of the bonding layer 15 is defined by an abutment position of the bump 22B and the recessed portion H10B.

The image pickup module 1B does not have “play” in the abutment position of the bump 22B and the recessed portion H10B, so that the cover glass 10 is positioned to a more accurate position than in the image pickup module 1.

### <Modification 3>

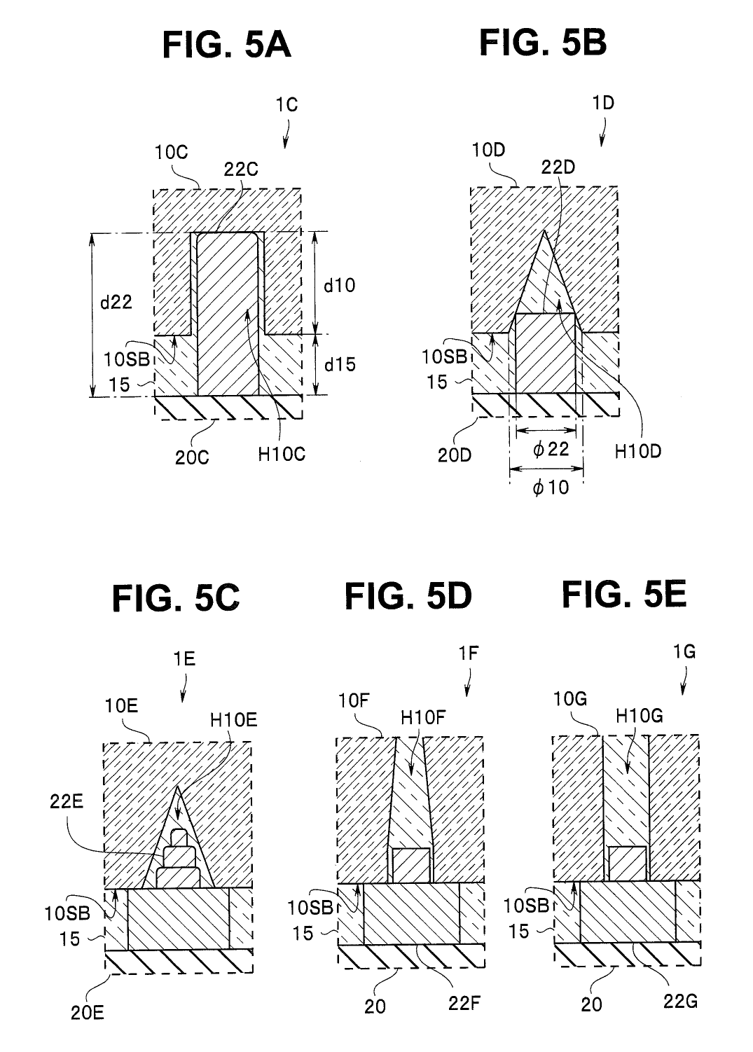

In the image pickup modules 1, 1A and 1B, in all of the bumps of the image pickup devices, the external dimensions of the lower portions are larger than the external dimensions of the upper portions. In contrast with this, in an image pickup module 1C of modification 3 shown in FIG. 5A, a bump 22C of an image pickup device 20C is a so-called straight bump in which an external dimension does not change in a height direction.

The external dimension of the bump 22C is smaller than an internal dimension of a recessed portion H10C. A height d22 of the bump 22C is longer than a depth d10 of the recessed portion H10C.

Consequently, when the bump 22C is inserted into the recessed portion H10C, a side surface of the bump 22C and a side surface of the recessed portion H10C face each other via a predetermined play, and a top surface of the bump 22C abuts on a bottom surface of the recessed portion H10C. That is, only an upper portion of the bump 22C is inserted into the recessed portion H10C. A difference between the height d22 of the bump 22C and the depth d10 of the recessed portion H10C corresponds to the thickness d15 of the bonding layer 15. In the image pickup module 1C, the thickness d15 of the bonding layer 15 can be also considered to be defined by the top surface position of the bump 22C, that is, an abutment position of the bump 22C and the recessed portion H10C.

The bump 22C of the image pickup module 1C is easier to manufacture than the multistage bumps 22 and 22A and the inclined bump 22B.

### <Modification 4>

In the image pickup modules 1, and 1A to 1C, the recessed portions are each in a cylindrical shape in which the internal dimension of the opening portion and the internal dimension of an inside are same dimensions. In contrast, in an image pickup module 1D of modification 4 shown in FIG. 5B, a recessed portion H10D is in a conical shape in which an internal dimension of an inside becomes continuously smaller than an internal dimension of an opening portion.

A bump 22D of an image pickup device 20D is a straight bump. An outside diameter φ22 of the bump 22D is smaller than an opening diameter φ10 of the recessed portion H10D.

Consequently, when the bump 22D is inserted into the recessed portion H10D, the bump 22D abuts on and is firmly fixed to the recessed portion H10D at a position where an outside diameter of the bump 22D becomes the same as an inside diameter of the recessed portion H10D. That is, the thickness d15 of the bonding layer 15 is defined by an abutment position of the bump 22D and the recessed portion H10D.

The image pickup module 1D does not have “play” in the abutment position, so that the cover glass 10D is positioned and fixed to a more accurate position.

### <Modification 5>

In an image pickup module 1E of modification 5 shown in FIG. 5C, a recessed portion H10E of a cover glass 10E is conical. A bump 22E of an image pickup device 20E is a multistage bump with a configuration analogous to the configuration of the bump 22A.

In the bump 22E, an external dimension of a lower bump is larger than an internal dimension of an opening portion, and an external dimension of an upper bump is smaller than the internal dimension of the opening portion. Consequently, when the bump 22E is inserted into the recessed portion H10E of the cover glass 10E, a top surface of the lower bump and the back surface 10SB of the cover glass 10E abut on each other.

Note that the thickness of the bonding layer 15 may be defined by a side surface of the upper bump of the bump 22E abutting on an inner surface of the recessed portion H10E without generating “play”. Further, the top surface of the lower bump and the back surface 10SB of the cover glass 10E may abut on each other, and the side surface of the upper bump and the inner surface of the recessed portion H10E may abut on each other at the same time.

### <Modifications 6 and 7>

In an image pickup module 1F in modification 6 shown in FIG. 5D and an image pickup module 1G in modification 7 shown in FIG. 5E, bumps 22F and 22G are two-stage bumps which are substantially the same as the bump 22.

In the image pickup module 1F, a recessed portion H10F of a cover glass 10F has a smaller internal dimension of an inside than an internal dimension of an opening portion as the recessed portion H10D of the image pickup module 1D of modification 4, but is a through-hole that penetrates through the cover glass 10F.

In the image pickup module 1G, a recessed portion H10G of a cover glass 10G is in a columnar shape similarly to the recessed portion H10 of the image pickup module 1, but is a through-hole that penetrates through the cover glass 10G. That is, a recessed portion formed in the cover glass may be a through-hole.

Note that in the image pickup modules 1F, 1G and the like, the adhesive agent of the bonding layer 15 does not have to fill the through-holes completely to upper portions.

### <Modification 8>

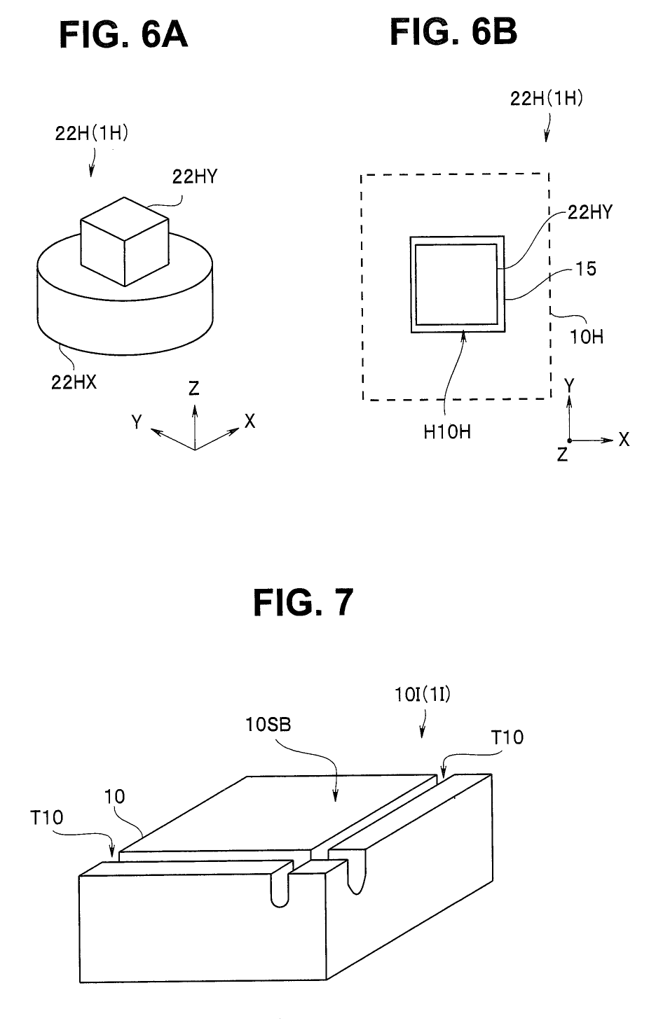

Next, a bump 22H of an image pickup device 20H of an image pickup module 1H of modification 8 shown in FIGS. 6A and 6B is a two-stage bump similarly to the bump 22. However, an upper bump 22HY placed on a lower bump 22HX is a rectangular parallelepiped with a rectangular sectional shape. A recessed portion H10H of a cover glass 10H is also rectangular in sectional shape.

When the upper bump 22HY of the rectangular parallelepiped is inserted into the recessed portion H10H rectangular in section, a relative position in three axial directions, that is, an in-plane direction (an XY-direction) and the vertical direction (Z-direction) is defined by a pair of the upper bump 22HY and the recessed portion H10H.

Consequently, at least one recessed portion H10H is formed in the cover glass 10H, and at least one bump 22H is placed on the image pickup device 20H.

Note that a sectional shape of a recessed portion and a sectional shape of an upper portion of a bump that is inserted into the recessed portion may be polygonal shapes such as a triangle and a hexagon, a cross and the like, if only the image pickup device and the cover glass can be uniquely defined in the in-plane direction.

### <Modification 9>

In a cover glass 10I of an image pickup module 1I of modification 9 shown in FIG. 7, two grooves T10 orthogonal to each other are formed by half-cut dicing or the like, a positioning bump of an image pickup device is inserted into the grooves T10, and positioning is performed. The two grooves may be formed to be parallel with each other, or three or more grooves may be formed in accordance with disposition of a plurality of positioning bumps.

The groove 10T is easier to form than a recessed portion.

### <Modifications 9, 10 and 11>

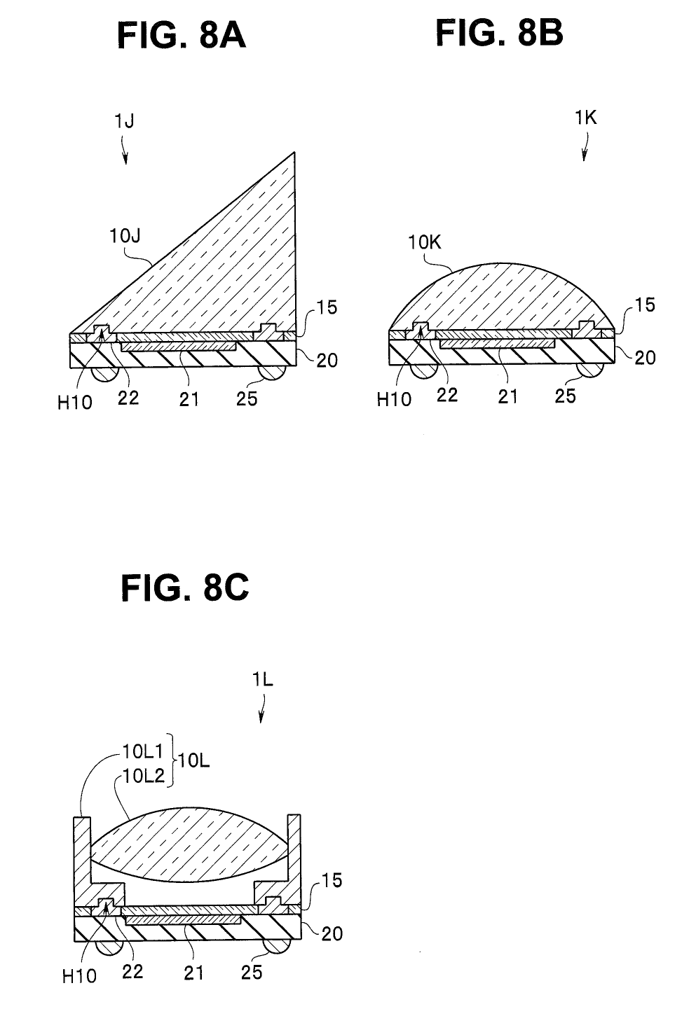

In an image pickup module 1J of modification 9 shown in FIG. 8A, an optical member bonded to the image pickup device 20 is a horizontal type of prism 10J. In an image pickup module 1K of modification 10 shown in FIG. 8B, an optical member bonded to the image pickup device 20 is a lens 10K. In an image pickup module 1L of modification 11 shown in FIG. 8C, an optical member bonded to the image pickup device 20 is an optical unit 10L having a lens 10L2 that is a transparent optical member and a frame member 10L1.

That is, the optical member which is bonded to the image pickup device is not limited to a transparent member such as a cover glass, but if the optical member is a frame-shaped spacer or the like that is placed under the cover glass, positioning can be accurately performed if only the positioning bump of the image pickup device can be inserted into the recessed portion.

### Second Embodiment

An image pickup module 1M of a second embodiment will be described next. The image pickup module 1M is analogous to the image pickup modules 1, 1A to 1L (the image pickup module 1 and the like), and has a same effect as the effects of the image pickup module 1 and the like, so that the same components will be assigned with the same reference signs, and explanation will be omitted.

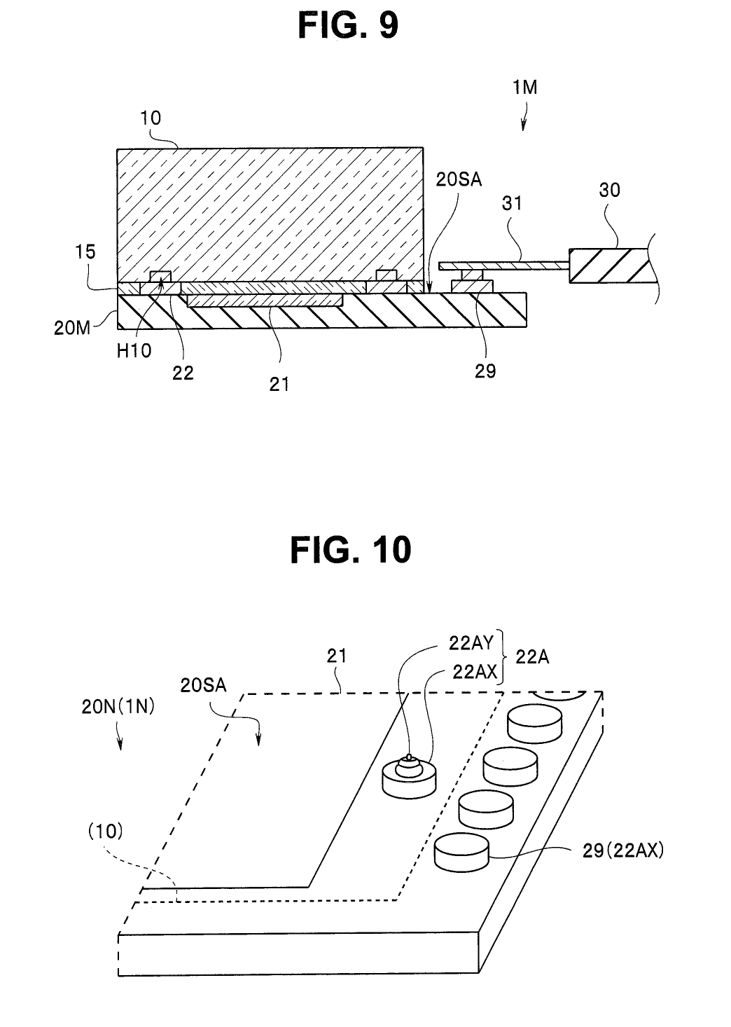

As shown in FIG. 9, the image pickup module 1M further includes a wiring board 30, in addition to the components included by the image pickup module 1 and the like.

The wiring board 30 has a plurality of flying leads 31 protruded from an end surface. The flying lead 31, which is also called an inner lead in a lead frame, is a rod-shaped metal conductor formed by selectively detaching an insulation layer and the like around wiring of the wiring board 30. For example, the flying lead 31 has a length of 250 μm, a thickness of 20 μm and a width of 50 μm. The wiring board 30 may be a double-sided wiring board, a multilayer wiring board or a component incorporating wiring board.

On an image pickup device 20M of the image pickup module 1M, a plurality of conducting bumps 29 that are electrically connected to the light receiving portion 21 are placed in a region that is not covered with the cover glass 10, of a light receiving surface 20SA.

The flying lead 31 of the wiring board 30 and the conducting bumps 29 of the image pickup device 20M are ultrasound-bonded, for example. The wiring board 30 is bonded to a signal cable though not illustrated, and the image pickup device 20M transmits and receives electric signals via the wiring board 30.

In the image pickup module 1M, the conducting bumps 29, and the two positioning bumps 22 each with an upper portion inserted into the recessed portion H10 of the cover glass 10 are simultaneously placed with configurations in which shapes, sizes and materials are same.

The conducting bump 29 is also an essential component in the conventional image pickup module, for external connection. The image pickup module 1M in which the positioning bump 22 and the conducting bump 29 are of the same configuration is substantially the same as the conventional image pickup module in manufacturing process, but it is easy to accurately position and bond the cover glass 10 by the positioning bumps 22.

Note that in the second embodiment, similar effects also can be exhibited by using the positioning bumps, the recessed portions and the optical members of the configurations of the modifications of the first embodiment.

### <Modification of Second Embodiment>

An image pickup module 1N of a modification of the second embodiment will be described next. The image pickup module 1N is analogous to the image pickup module 1M, and has a same effect as the effect of the image pickup module 1M, so that same components will be assigned with same reference signs and explanation will be omitted.

As illustrated in FIG. 10, in the image pickup module 1N, a positioning bump 22A of an image pickup device 20N is a multi-stage bump in which an upper bump 22AY is placed on a top surface of a lower bump 22AX of a same configuration as the configuration of the conducting bump 29. For example, in the image pickup module 1N, the conducting bumps 29 and the lower bump 22AX are plated bumps, and the upper bump 22AY is a gold stud bump.

The image pickup module 1N is easy to manufacture because the bump 22A for positioning is placed in a same step as the conducting bump 29.

### Third Embodiment

An endoscope 2 of a third embodiment will be described next.

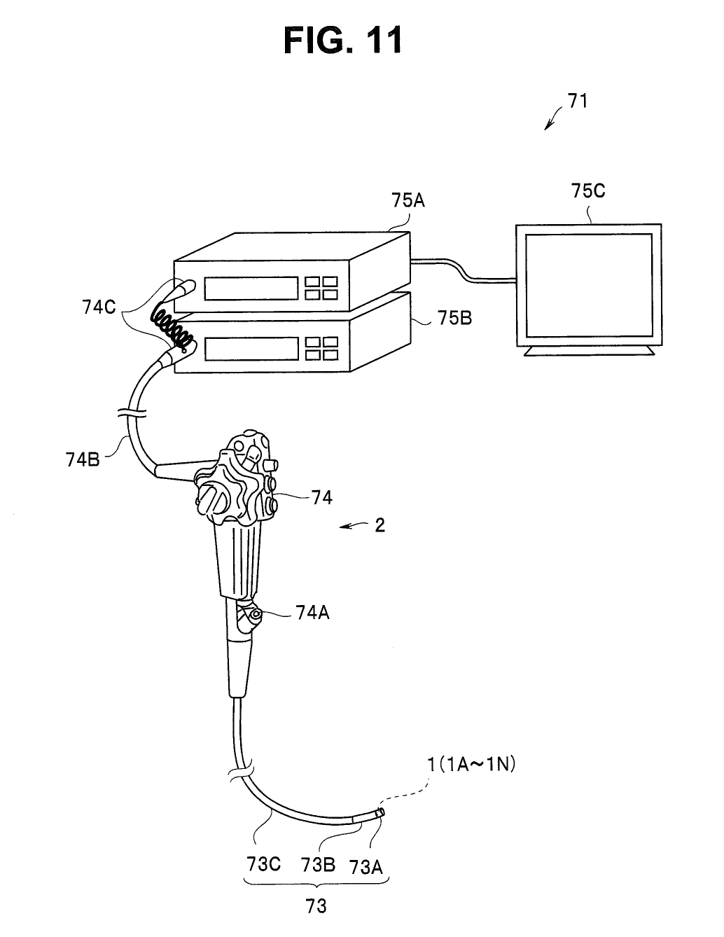

As shown in FIG. 11, an endoscope system 71 includes an endoscope 2, a processor 75A, a light source apparatus 75B and a monitor 75C. The endoscope 2 inserts an insertion portion 73 into a body cavity of a subject, and thereby picks up an intracorporeal image of the subject to output an image pickup signal.

At a proximal end side of the insertion portion 73 of the endoscope 2, an operation portion 74 provided with various buttons that operate the endoscope 2 is placed. In the operation portion 74, a treatment instrument insertion port 74A of a channel 73H for inserting a treatment instrument such as biological forceps, an electric knife and an inspection probe into a body cavity of the subject are located.

The insertion portion 73 is configured by a distal end portion 73A in which the image pickup module 1 is placed, a bending portion 73B that is connectively provided at a proximal end side of the distal end portion 73A and is bendable, and a flexible tube portion 73C connectively provided at a proximal end side of the bending portion 73B. The bending portion 73B is bent by an operation of the operation portion 74.

A signal cable 75 connected to the image pickup module 1 in the distal end portion 73A is inserted through a universal cord 74B placed at a proximal end portion side of the operation portion 74.

The universal cord 74B is connected to the processor 75A and the light source apparatus 75B via connectors 74C. The processor 75A controls the entire endoscope system 71, and performs signal processing to an image pickup signal that is outputted by the image pickup module 1 and outputs the image pickup signal as an image signal. The monitor 75C displays the image signal outputted by the processor 75A.

The endoscopes 2, which have the image pickup modules 1 and 1A to 1N in which the optical members are accurately positioned and bonded to the light receiving surfaces of the image pickup devices, in the distal end portions 73A, are easy to manufacture.

Note that the endoscope of the embodiment is not limited to a flexible endoscope including the flexible tube portion 73C, but may be a rigid endoscope, or may be a capsule type endoscope.

The present invention is not limited to the aforementioned embodiments, modifications and the like, but various modifications, combinations and applications can be made within the range without departing from the gist of the invention.

What is claimed is:

1. An endoscope including an image pickup module in a distal end portion of an insertion portion,
wherein the image pickup module comprises
an optical member, and
an image pickup device to which the optical member is bonded via a bonding layer to cover a light receiving portion so that light is incident on the light receiving portion via the optical member,
a positioning bump is placed in a peripheral portion of the light receiving portion of the image pickup device,
a recessed portion is formed in the optical member, and
an upper portion of the positioning bump of the image pickup device is inserted into the recessed portion of the optical member, and a relative position in three axial directions of the image pickup device and the optical member is defined by an abutment position of the positioning bump and the recessed portion.

2. An image pickup module, comprising:
an optical member; and
an image pickup device to which the optical member is bonded via a bonding layer to cover a light receiving portion so that light is incident on the light receiving portion via the optical member,
wherein a positioning bump is placed in a peripheral portion of the light receiving portion of the image pickup device,
a recessed portion is foliated in the optical member, and
an upper portion of the positioning bump of the image pickup device is inserted into the recessed portion of the optical member, and a relative position in three axial directions of the image pickup device and the optical member is defined by an abutment position of the positioning bump and the recessed portion.

3. The image pickup module according to claim 2,
wherein in the positioning bump, an external dimension of the upper portion is smaller than an external dimension of a lower portion.

4. The image pickup module according to claim 2,
wherein in the recessed portion, an internal dimension of an opening portion is smaller than an internal dimension of an inside.

5. The image pickup module according to claim 2,
wherein in the positioning bump, a sectional shape of the upper portion is circular,
in the recessed portion, a sectional shape is circular,
the positioning bump in plurality are placed on the image pickup device, and
the recessed portion in plurality are formed in the optical member.

6. The image pickup module according to claim 5,
wherein the recessed portion is a groove.

7. The image pickup module according to claim 2,
wherein sectional shapes of the upper portion of the positioning bump and the recessed portion are rectangular.

8. The image pickup module according to claim 2,
wherein the optical member is an optical unit including a cover glass, a lens, a prism or a transparent optical member and a frame member.

9. The image pickup module according to claim 2, further comprising:
a wiring board in which a plurality of flying leads are protruded from an end surface,
wherein a plurality of conducting bumps that are electrically connected to the light receiving portion are placed in a region that is not covered with the optical member, in the peripheral portion of the light receiving portion of the image pickup device, and
the conducting bumps and the flying leads are bonded.

10. The image pickup module according to claim 9,
wherein a configuration of the positioning bump and a configuration of each of the conducting bumps are same.

11. The image pickup module according to claim 9,
wherein the positioning bump is a multistage bump in which an upper bump that is inserted into the recessed portion is placed on a top surface of a lower bump of a same configuration as the configuration of each of the conducting bumps.

12. A manufacturing method of an image pickup module, comprising:
a step of producing an optical member in which recessed portions are formed, and an image pickup device in which a plurality of positioning bumps are placed on a peripheral portion of a light receiving portion;
a step of a relative position in three axial directions of the image pickup device and the optical member being positioned by inserting upper portions of the positioning bumps of the image pickup device into the recessed portions of the optical member; and
a step of curing an adhesive agent interposed between the optical member and the image pickup device, in a positioned state.
